(19) United States
(12) Patent Application Publication
(10) Pub. No.: US 2014/0243778 A1
(43) Pub. Date: Aug. 28, 2014

(54) GARMENT SEAM POSITIONING AND CONSTRUCTION

(71) Applicant: Ronald Boser, Port St. Lucie, FL (US)

(72) Inventor: Ronald Boser, Port St. Lucie, FL (US)

(21) Appl. No.: 14/186,590

(22) Filed: Feb. 21, 2014

Related U.S. Application Data
(60) Provisional application No. 61/769,497, filed on Feb. 26, 2013.

(51) Int. Cl. A41D 27/24; A61F 13/66; A41B 9/04

(52) U.S. Cl. CPC A41D 27/245; A41B 9/04; A61F 13/665
USPC 604/396; 2/406; 112/475.09

(57) ABSTRACT

Disclosed herein are undergarments having a seam in a crotch region thereof and methods of manufacturing such undergarments. A seam in the undergarments is formed by joining first and second sections of material each having an inner surface and an outer surface and a perimeter about the inner and outer surfaces thereof. A portion of an inner surface of a panty liner contacts a portion of the inner surfaces of the first and second sections of material and the seam. The location of the seam is such that when the undergarment is worn by a user, the seam cannot be seen by viewing the front side or back side of the user.

### CROSS-REFERENCE TO RELATED APPLICATIONS

The present application claims the benefit of the filing date of U.S. Provisional Patent Application No. 61/769,497 filed Feb. 26, 2013, the disclosure of which is hereby incorporated herein by reference.

### FIELD OF THE INVENTION

The present invention relates to positioning of a seam in undergarments and in particular relates to positioning of the seam such that when the undergarment is worn by a user, the seam cannot be seen by viewing the front side or back side of the user.

### BACKGROUND OF THE INVENTION

Construction of undergarments such as underwear generally takes into consideration comfort and style for the user. Many undergarments are manufactured from two or more pieces of material that need to be joined together in some manner. A seam is generally formed at the junction of the two or more pieces of material. In most cases, the seam is created by stitching the pieces of material together. Depending on the desired look and/or quality of the undergarment, many different stitching methods or patterns may be used.

Additional seams may be created by adding one or more bands in order to keep the undergarment positioned in a certain area of the body of a user. Lace and other decorative materials may also be joined around the perimeter of the joined materials. Depending on the construction and function of the undergarment, many seams may be created in the manufacturing thereof. These seams may be unsightly depending on their quality and location on the undergarment. A seam that can be viewed from the front or back side of a user is generally not a preferred design.

In addition to the joining of different pieces of material together to form active wear, bathing suits and undergarments, most female underwear also includes an underwear lining or a panty liner. A panty liner is an absorbent piece of material used for feminine hygiene. Panty liners are found in an assortment of sizes, shapes, scents and portability options, ranging from tiny, compact liners to long, protective liners.

### BRIEF SUMMARY OF THE INVENTION

The present invention is a novel design of an undergarment in which the seam used to join two separate sections or pieces of the undergarment material together cannot be viewed from either the front side or back side of the user. A panty liner is then assembled to the joined pieces of material at least overlying a portion of the seam.

A first aspect of the present invention is an undergarment including a first section of material, a second section of material and a panty liner. The first section of material has an inner surface and an outer surface and a perimeter about the inner and outer surfaces thereof. The second section of material has an inner surface and an outer surface and a perimeter about the inner and outer surfaces thereof. The panty liner has an inner surface and an outer surface and a perimeter about the inner and outer surfaces thereof. A portion of the inner surface of the panty liner contacts a portion of the inner surfaces of the first and second sections of material at a seam in a crotch region of the undergarment created by the joining of the first section of material to the second section of material. The seam is preferably adjacent a portion of the perimeters of the first and second sections of materials.

A second aspect of the present invention is a method of manufacturing an undergarment including first and second sections of material and a panty liner each having inner and outer surfaces and perimeters about the inner and outer surfaces thereof. The method includes forming a seam in a crotch region of the undergarment by joining a portion of an inner surface of the second section of material to an outer surface of the first section of material and joining the panty liner with the first and second joined pieces of material with adhesive by covering at least a portion of the seam with an inner surface of the panty liner.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a perspective view of a prior art undergarment.

FIG. 2 is a plan view of a portion of the undergarment shown in FIG. 1 adjacent the crotch region of the undergarment.

FIG. 3 is a cross-sectional view of the undergarment in FIG. 2 taken along line 3-3 thereof.

FIG. 4 is one embodiment of a portion of an undergarment of the present invention.

FIG. 5 is a cross-sectional view of the undergarment in FIG. 4 taken along line 5-5 thereof.

FIG. 6 is a perspective view of folded first and second sections of material joined together forming a seam.

FIG. 7 is a perspective view of the joined first and second sections of material in FIG. 6 in an unfolded state.

FIG. 8 is a perspective view of the unfolded first and second sections of material of FIG. 7 wherein a securing means is applied to the seam.

### DETAILED DESCRIPTION

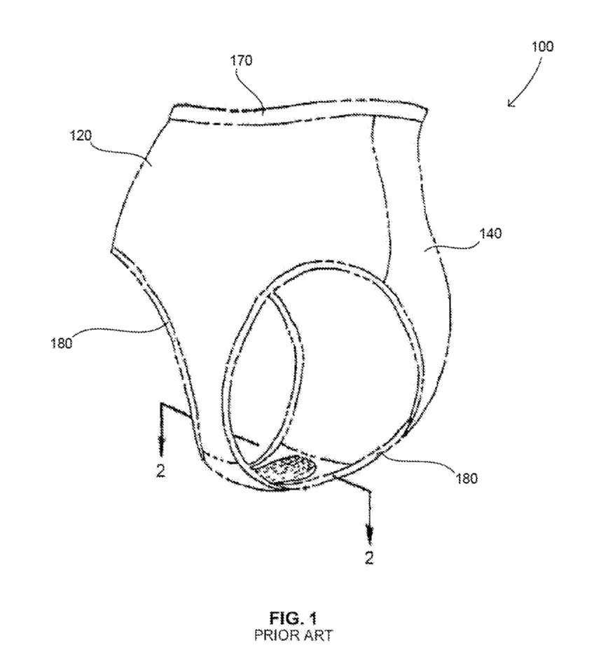

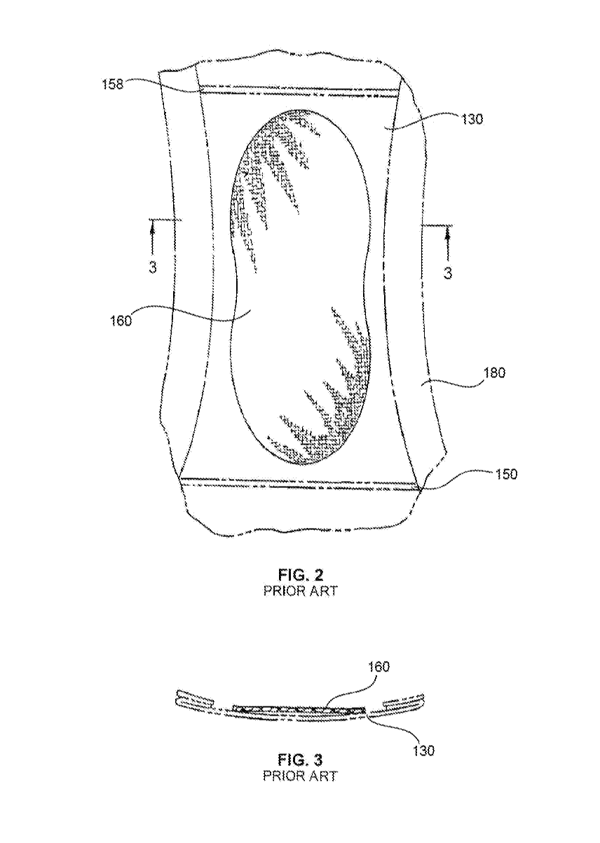

FIGS. 1-3 show an embodiment of a prior art undergarment 100. Undergarment 100 includes a first section or front panel 120, a second section or crotch panel 130 and a third section or rear panel 140 of material. A first seam 150 is created by the joining of the first section 120 and second section 130 of material. A second seam 158 is created by the joining of the first section 120 and second section 130. The locations of seams 150, 158 make it likely that when a user wears undergarment 100, seam 150 can be seen when viewing the front side of the user while seam 158 can be seen when viewing the back side of the user.

Further, a panty liner 160 is fastened to second section 130. When worn, second section 130 of undergarment 100 is generally located along what may be referred to as the sagittal plane of the user. Undergarment 100 further includes a waist band 170 that spans at least a portion of the perimeters of joined first section 120 and third section 140 and first and second thigh bands 180 each of which span at least a portion of the perimeters of joined first section 120 and second section 130 and joined second section 130 and third section 140.

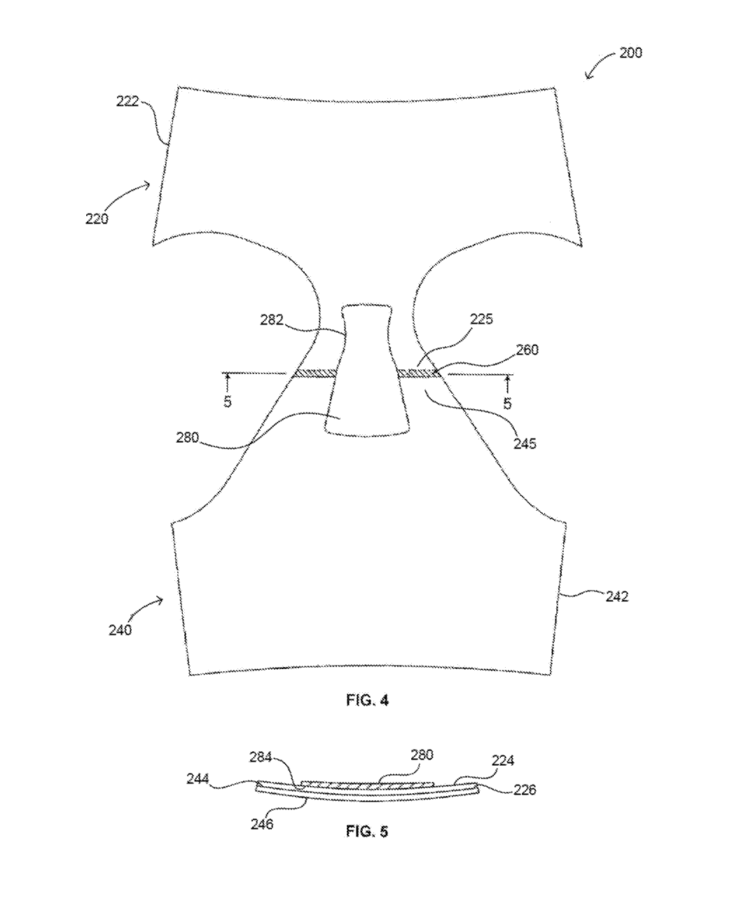

FIG. 4 is a plan view of an embodiment of a portion of an undergarment 200 of the present invention. The portion of undergarment 200 shown includes a first section or front panel 220 and a second section or rear panel 240 of material each having perimeters 222 and 242 respectively. A portion of perimeter 222 of first section 220 is joined to a portion of perimeter 242 of second section 240 forming a seam 260 adjacent a crotch region of undergarment 200. When undergarment 200 is being worn, seam 260 cannot be seen when viewing undergarment 200 from either a front side or a back side thereof.

Preferably, adhesive is applied to an inner surface 244 of second section 240 along a portion of perimeter 242 adjacent a narrowing section 245 thereof. An outer surface 226 of first section 220 along a portion of perimeter 222 adjacent a narrowing section 225 thereof is brought in contact with the adhesive on the inner surface 244 of second section 240. The adhesive joins the first section 220 to the second section 240 at seam 260. Alternatively, first section 220 and second section 240 may be joined by stitching or ultrasonic welding of seam 260 at the same crotch region.

An inner surface 284 of panty liner 280 including adhesive thereon is then brought into contact with inner surface 224 of first section and inner surface 244 of second section 240 as shown in FIG. 5. Also shown is an outer surface 226 of first section 220 and an outer surface 246 of second section 240. The portion of panty liner 280 that overlies seam 260 is preferably an intermediate portion of panty liner 280. Perimeter 282 of panty liner 280 cannot be seen when viewing undergarment 200 from either a front side or a back side thereof. In other embodiments, adhesive is applied adjacent seam 260 and narrowing sections 225, 245 of inner surfaces 224, 244 of first and second sections 220, 240 respectively, and inner surface 284 of panty liner 280 is then brought in contact with the first and second sections 220, 240 having adhesive applied thereto in order to join panty liner 280 with first and second sections 220, 240.

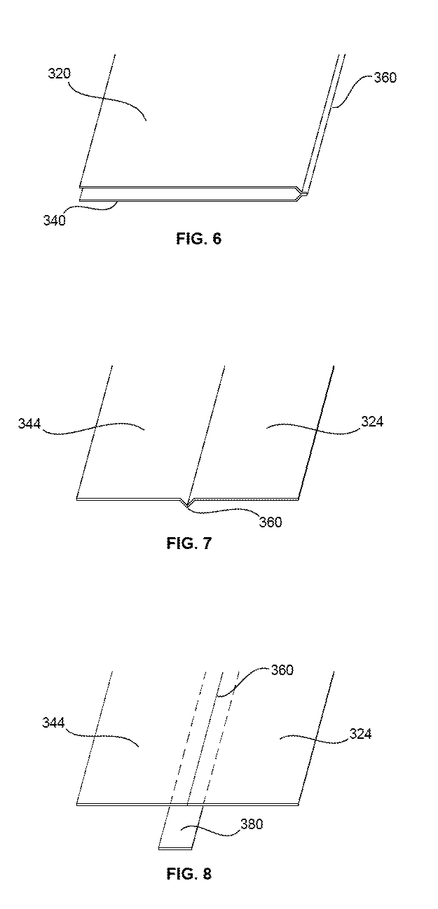

FIG. 6 is a plan view of a first second of material 320 folded with respect to a second section of material 340 such that inner surfaces 324, 344 of first and second sections of material 320, 340 respectively are facing one another. First and second sections of material 320, 340 are joined together at side edges thereof forming a seam 360. Preferably, seam 360 is created by joining the first and second sections of material 320, 340 together by ultrasonic welding. The ultrasonic welding causes the materials 320, 340 to adhere together in such a manner that a substantially straight joint line is formed. First and second sections of material 320, 340 may also be joined together by stitching and/or adhesive.

FIG. 7 is a perspective view of the joined first and second sections of material 320, 340 in FIG. 6 wherein the first and second sections of material 320, 340 are unfolded exposing the inner surfaces 324, 344 of first and second sections of material 320, 340 and an inner surface of seam 360. Seam 360 is shown slightly below the plane of joined materials 320, 340 when in an initial unfolded state.

As shown in FIG. 8, the first and second sections of material 320, 340 are flattened such that inner surfaces 324, 344 and seam 360 now lie substantially on the same plane. A securing means is then applied to seam 360. The securing means is preferably a combination of an adhesive and binder strip 380 to secure seam 360. The adhesive 380 may be sprayed on seam 360 and adjacent portions of first and second sections of material 320, 340. Adhesive may be first applied to an inner surface of binder strip 380 before contact is made with seam 360 and adjacent portions of first and second sections of material 320, 340. Alternatively, the adhesive and/or binder strip 380 may be applied, rolled, or brushed on to seam 360 and adjacent portions of first and second sections of material 320, 340.

The binder strip 380 is applied to inner surfaces 324, 344 of first and second sections of material 320, 340 such that inner surfaces 324, 344 and the binder strip lie substantially on the same plane as shown in FIG. 8. This seam construction provides more comfort during wear for the user and is less noticeable than standard sewn seams, for example.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-2. (canceled)

3. An undergarment comprising:
a first section of material having an inner surface and an outer surface and a perimeter about the inner and outer surfaces thereof;
a second section of material having an inner surface and an outer surface and a perimeter about the inner and outer surfaces thereof;
a seam formed by joining the first and second sections of material; and
a panty liner having an inner surface and an outer surface,
wherein a portion of the inner surface of the panty liner contacts a portion of the inner surfaces of the first and second sections of material and the seam.

4. The undergarment of claim 3, wherein the seam is formed by joining the first section of material to the second section of material adjacent a portion of the perimeters thereof.

5. The undergarment of claim 4, wherein the seam is formed by joining the outer surface of the first section of material to the inner surface of the second section of material.

6. The undergarment of claim 5, wherein the first section of material is joined to the second section of material at the seam by stitching.

7. The undergarment of claim 5, wherein the first section of material is joined to the second section of material at the seam by adhesive.

8. The undergarment of claim 4, wherein the seam is formed by joining the inner surface of the first section of material to the outer surface of the second section of material.

9. The undergarment of claim 8, wherein the first section of material is joined to the second section of material at the seam by stitching.

10. The undergarment of claim 8, wherein the first section of material is joined to the second section of material at the seam by adhesive.

11. The undergarment of claim 3, wherein the seam is form in a crotch region of the undergarment.

12. An undergarment comprising:
a first section of material having an inner surface and an outer surface and a perimeter about the inner and outer surfaces thereof;
a second section of material having an inner surface and an outer surface and a perimeter about the inner and outer surfaces thereof;
a seam formed by joining the first and second sections of material adjacent a portion of the perimeters thereof; and
a panty liner having an inner surface and an outer surface,
wherein the inner surface of the panty liner contains an adhesive applied thereto such that when the inner surface of the panty liner contacts a portion of the inner surfaces of the first and second sections of material and the seam, the panty liner is fastened to the inner surfaces of the first and second sections of material and the seam.

13. The undergarment of claim 12, wherein the seam is formed by joining the outer surface of the first section of material to the inner surface of the second section of material.

14. The undergarment of claim 12, wherein the seam is formed by joining the inner surface of the first section of material to the outer surface of the second section of material.

15. A method of manufacturing an undergarment including first and second sections of material and a panty liner each having inner and outer surfaces and perimeters about the inner and outer surfaces thereof comprising:
forming a seam in a crotch region of the undergarment by joining the first and second sections of material; and
covering at least a portion of the of the inner surfaces of the first and second sections of material and the seam with the panty liner.

16. The method of claim 15, wherein the seam is formed by joining the first section of material to the second section of material adjacent a portion of the perimeters thereof.

17. The undergarment of claim 16, wherein the seam is formed by joining the outer surface of the first section of material to the inner surface of the second section of material.

18. The undergarment of claim 17, wherein the first section of material is joined to the second section of material at the seam by stitching.

19. The undergarment of claim 17, wherein the first section of material is joined to the second section of material at the seam by adhesive.

20. The undergarment of claim 16, wherein the seam is formed by joining the inner surface of the first section of material to the outer surface of the second section of material.

21. The undergarment of claim 20, wherein the first section of material is joined to the second section of material at the seam by stitching.

22. The undergarment of claim 20, wherein the first section of material is joined to the second section of material at the seam by adhesive.
